(19) United States
(12) Patent Application Publication
(10) Pub. No.: US 2026/0020912 A1
(43) Pub. Date: Jan. 22, 2026

(54) Patient-Specific Marking Guide and Method of Design and Use in Endoscopic Craniosynostosis Surgery

(71) Applicant: KLS Martin, L.P., Jacksonville, FL (US)

(72) Inventors: Brett Walter, Jacksonville, FL (US); Erica Wessner, Jacksonville, FL (US)

(73) Assignee: KLS Martin, L.P., Jacksonville, FL (US)

(21) Appl. No.: 19/273,669

(22) Filed: Jul. 18, 2025

Related U.S. Application Data
(60) Provisional application No. 63/673,139, filed on Jul. 18, 2024.

(51) Int. Cl. A61B 34/10; A61B 17/00; A61B 17/32; A61B 17/3211

(52) U.S. Cl. CPC A61B 34/10; A61B 17/3211; A61B 2017/00526; A61B 2017/320052; A61B 2034/105; A61B 2034/108

(57) ABSTRACT

A patient-specific marking guide for marking or guiding the incision in endoscopic craniosynostosis to correct for prematurely fused sutures, and the method of its creation and use wherein the marking guide has an upper portion with one or more windows and one or two lower conforming portions that match the configuration of the patient's ear or ears, such that with the lower conforming portion or portions mounted onto the ear or ears of the patient, the window or windows will be precisely located to mark the desired location and trajectory for the incision or incisions.

### BACKGROUND OF THE INVENTION

This invention relates to treatments and instruments used in endoscopic craniosynostosis surgery, wherein a prematurely fused cranial suture in an infant is separated to allow for proper brain growth. A newborn's skull is made up of many separate bones that are normally unfused such that the brain can grow. Sutures or anatomical lines are strong, fibrous tissue joining the bony plates of the skull. Complete fusion of the bones takes many years, typically occurring in the teen years. Premature fusion can occur between two bones or between multiple bones. In endoscopic craniosynostosis surgery, the surgeon uses an endoscope inserted through a relatively small incision to visualize and sever the fused suture.

It is important that the surgeon properly locate the incision site or sites relative to the location and orientation of the fused suture or sutures, but this is complicated by the fact that soft tissue covers the fused suture site or sites. It is an object of this invention to provide a patient-specific marking guide which conforms to the shape and configuration of the soft tissue and which is mounted onto an external anatomical feature or prominence, such as an ear or nose, the marking guide providing a window or edge for the surgeon to mark on the patient's soft tissue, i.e., skin, that precisely indicates the trajectory and position of the fused suture. It is a further object to provide such a guide using imaging of the patient's head, such as for example CT scanning, to create a 3-D virtual model of both the fused suture and the external configuration of the soft tissue, including the external anatomical feature.

### SUMMARY OF THE INVENTION

The invention in various embodiments comprises a patient-specific, i.e., customized, marking guide which indicates to the surgeon the proper location on the patent's head for making the incision to obtain access endoscopically to the fused suture for separation. The marking guide preferably comprises an elongated opening or window positioned on the upper portion of the marking guide that conforms to the outer surface configuration of the patient's head. The lower portion of the marking guide defines a registration portion, the registration portion being designed to conform or mount onto at least a portion of an external anatomical feature or prominence of the head of the patient, such as an ear or nose, whereby the registration portion and therefore the marking guide itself will only mount onto the external anatomical feature in a single, desired orientation. For example, for a marking guide conforming to an ear, the lower registration portion may comprise an upper curved segment configured to rest on the tissue above the cartilage between the helix of the ear and the temporal skin and a lower base segment configured to reside within the concha of the ear and over the ear canal, the lower base segment having a convex configuration that mates or matches the concave concha. In this manner the irregular shape of the concha and the lower base segment ensure that there is only a single orientation whereby the lower base segment fits in mated manner with the concha. The marking guide in an alternative embodiment may extend completely across the head and comprise a lower registration portion for each ear. Likewise, the upper portion may comprise multiple marking windows.

The invention is also the method of designing and utilizing the marking guide such that is specific to an individual patient. A virtual 3-D model of the patient's cranium and of the external soft tissue of the head is created by CT-scan or similar imaging equipment. The creation of virtual 3-D images for surgical planning and the creation of implants is well known in the art. In consultation with the surgeon, the location and trajectory of the fused suture is determined. A technician designs a virtual marking guide for placement on the soft tissue surface, i.e., the skin, comprising an upper portion with a window corresponding to the desired incision location to provide access to the fused suture and a lower registration portion of the marking guide conforming to one or both ear's external anatomical features, namely the arch at the base of the helix and the concha. Based on this virtual design, an actual marking guide is manufactured. With the marking guide properly oriented onto the patient's ear the surgeon can mark the incision location. It is also possible to use the window of the marking guide as a boundary for guiding the scalpel as the incision is made.

### BRIEF DESCRIPTION OF THE DRAWINGS

FIG. 1 is a perspective view of an embodiment of the patient-specific marking guide, shown positioned on a virtual 3-D model of an infant's head.

FIG. 2 is a front view of the embodiment of FIG. 1.

FIG. 3 is a front view of alternative embodiment of the marking guide, shown positioned on a virtual 3-D model of a human head wherein two windows are provided and the marking guide extends completely across the head and registers with both ears.

FIG. 4 is a right side view of the embodiment of FIG. 3.

FIG. 5 is a left side view of the embodiment of FIG. 3.

### DETAILED DESCRIPTION OF THE INVENTION

With reference to the drawings, the invention will be described in detail with regard to the marking guide and the method of creation and use of the marking guide. It is to be understood that the drawings are for illustrative, enabling and definitional purposes, and are not meant to limit the scope of the invention. The terms upper or superior refer to the direction toward the top of the head and the terms lower and inferior refer to the opposite direction toward the bottom of the head. The term patient-specific defines the invention as being customized for individual patients, such that no two marking guides will be identical.

It is to be understood that in addition to the examples of unilateral coronal synostosis and bilateral coronal synostosis discussed below, the invention may address all types of craniosynostosis, including for example other single-suture synostoses such as sagittal, lambdoid or metopic, other double-suture synostoses such as bilambdoid or combined sagittal/metopic, and complex multi-suture synostoses such as combined bicoronal/sagittal/metopic, cloverleaf skull, or pancraniosynstosis.

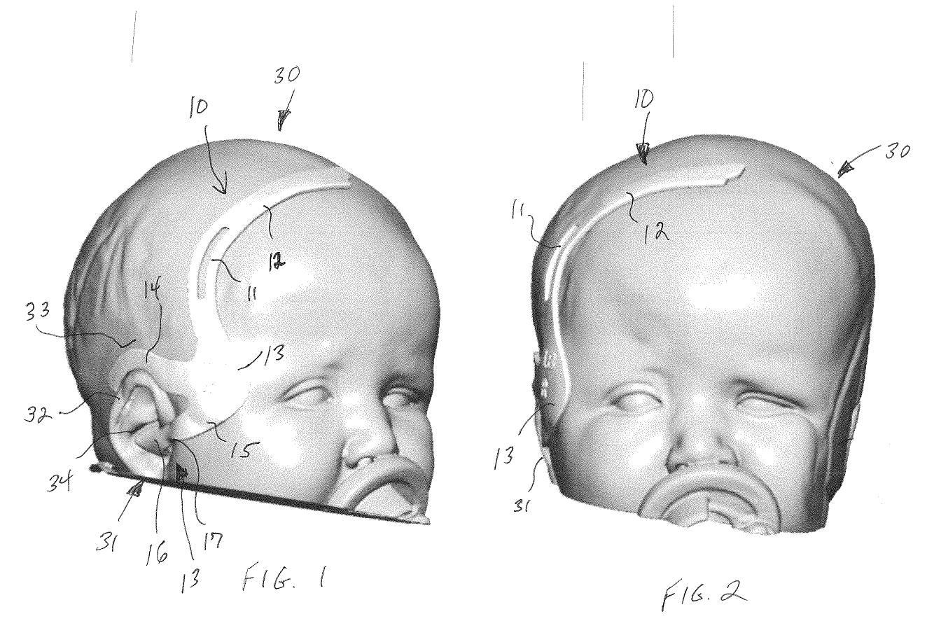

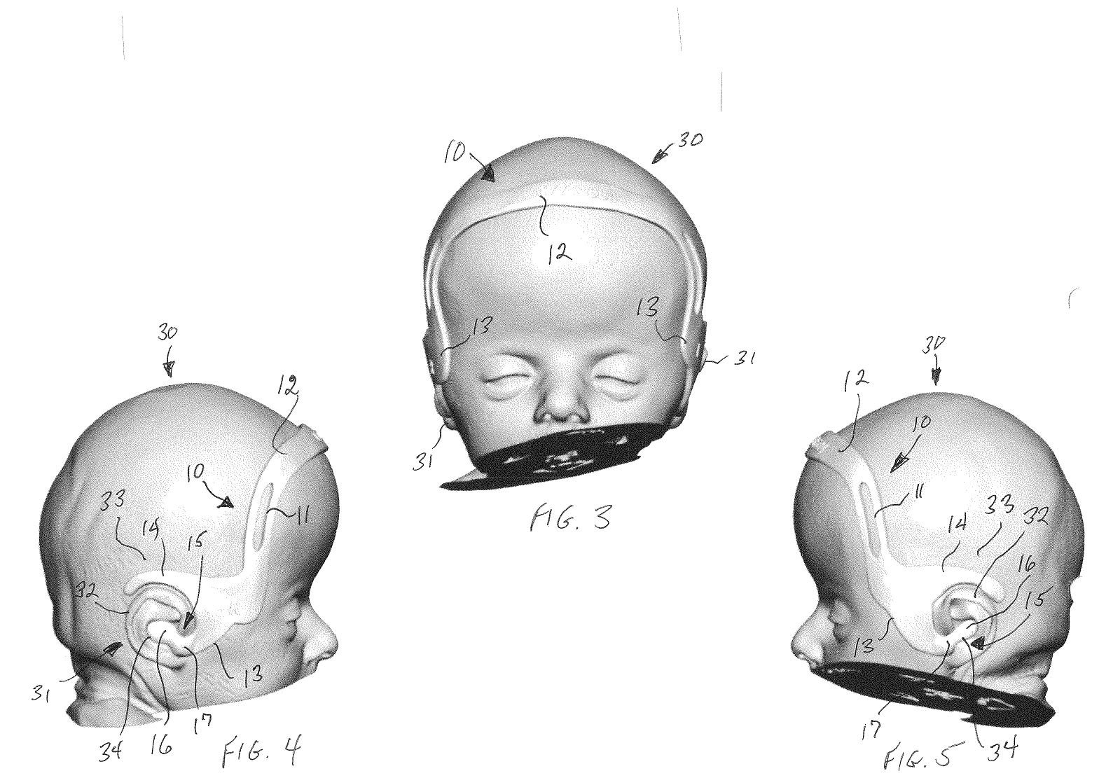

FIGS. 1 and 2 illustrate an embodiment of the patient-specific marking guide 10 for use with unilateral coronal synostosis, wherein a single side coronal suture is prematurely fused and needs to be surgically separated by endoscopic craniosynostosis surgery. FIGS. 3 through 5 illustrate an embodiment of the marking guide 10 for use with bilateral coronal synostosis, wherein the right side coronal suture and the left side coronal suture are prematurely fused and need to be surgically separated by endoscopic craniosynostosis surgery. In these examples, the external anatomical structure or feature used to ensure proper orientation of the marking guide 10 is the ear 31.

To produce the customized, patient-specific, marking guide 10, a patient's head 30 is imaged in known manner using a CT scanner or similar imaging equipment to produce a virtual 3-D model which shows the cranial bone structure and the fused suture or sutures. On the same or a different virtual 3-D model, the soft tissue configuration and surface contours of the external skin and external anatomical features or prominences, such as an ear or the nose, are visualized. The surgeon is then consulted to determine the desired location, shape and trajectories of the incision or incisions to correct the coronal synostosis, such that the incision or incisions is properly located over the fused suture.

Once the location and trajectory of the incision is determined, the technician designs a virtual marking guide 10 having an inner surface conforming to the outer surface configuration of the soft tissue 3-D model. The upper portion 12 of the marking guide 10 extends along one side of the cranial portion of the patient and is provided with an elongated aperture or window 11 in the form of a slot or elongated notch which corresponds to the predetermined incision location, as shown in FIGS. 1 and 2. In the case of bi-coronal synostosis, the upper portion 12 will extend fully across the cranium and two windows 11 will be provided, as shown in FIGS. 3-5.

The technician then designs the lower conforming registration portion 13 of the virtual marking guide 10, which is joined to the upper portion 12, to conform or mate with the anatomical external structure of the patient's ear or ears 31, in particular the concha 34 and the temporal junction between the helix 32 and the temporal skin 33 where the ear 31 is joined to the head, the conformation being such that the marking member 10 can only be mounted in one desired orientation relative to the ear or ears 31, thus ensuring that the upper portion 12 is properly located on the patient.

The lower conforming or registration portion 13 comprises two main segments. An upper curved segment 14 is configured to be positioned on the patient along the temporal junction of the ear 31 between the helix 32 and the temporal skin 33, i.e., at the location where the temporal arms of eyeglasses rest. A lower base segment 15 is shaped to fit into and mate with the concha 34, the hollow, concave, central part and largest cavity of the external ear 31, such that the configuration and contour of the convex inner surface of the lower base segment 15 matches the concave configuration and contour of the outer skin surface of the concha 34. The lower base segment 15, unlike Bluetooth earbuds for example which are configured in a standardized one-size-fits-all shape, fully mates in a nesting manner with the concha 34 such that the irregular configuration of the lower base segment 15 in combination with the upper curved segment 14 being snuggly seated on the upper ear junction guarantees that the lower registration portion 13 can only be mounted onto the patient in the one desired orientation. If the upper portion 12 of the marking guide 10 is improperly positioned, the lower base segment 15 will not mate with the concha 34.

In the case of a marking guide 10 extending from ear 31 to ear 31 across the cranium, this process is performed separately for each ear 31 of the patient, such that two lower base segments 15 will not be identical but will be customized for each ear 31.

The physical marking guide 10 is then produced from the design specs and parameters determined from the virtual 3-D model. The surgeon then places the marking guide 10 onto the patient by positioning the helix-adjacent upper curved segment 14 on the junction between the ear 31 and the temporal skin 33 and mating the lower conforming segment 15 of the marking guide 10 within the concha 34. In this manner, the window 11 of the upper portion 12 resides in the exact location for the desired incision. For bi-coronal synostosis, each lower base segment 15 is mated with its corresponding ear 31.

The surgeon then marks the location for the incision or incisions within the window or windows 11 and removes the marking guide 10 prior to performing the incision or incisions. Alternatively, the window or windows 11 may be used as physical boundaries for making the incision in the proper location.

An embodiment of a marking guide 10 as used for unilateral coronal synostosis is illustrated in FIGS. 1 and 2. In this embodiment the marking guide 10 comprises an elongated, strap-like, upper portion 12 having a window 11, the inner surface of the upper portion 12 conforming to the outer surface of the soft tissue of the patient's head. The upper portion 12 is attached to and extends from the lower registration portion 13. The upper curved segment 14 of the lower registration portion 13 extends forward and rearward between the helix 32 and temporal skin 33 so as to abut or rest on the junction formed between the ear 31 and temporal skin 33. At the forward end the upper curved segment 14 transitions into lower base segment 15. The inner surface of the lower base segment 15 is sized and configured to match the size and configuration of the outer surface of the concave concha 34, the deep recess or cavity forming the hollow central part of the ear 31. In this manner the convex lower base segment 15 is irregularly shaped with a thick, somewhat elongated, main body portion 16 having a forward extending short leg 17 which mates tightly and snuggly with the concha 34.

FIGS. 3 through 5 illustrate an alternative embodiment of the marking guide 10 for use in bilateral coronal synostosis, wherein the marking guide 10 extends from a first ear to a second ear across the cranium and has a pair of windows 11 to mark the locations of the incisions, or to be used a physical guide to make the incisions.

It is contemplated that equivalents or substitutions for elements set forth above may be obvious to those of skill in the art, and therefore the true scope and definition of the invention is to be as set forth in the following claims.

I claim:

1. A method of creating a patient-specific craniosynostosis marking guide comprising the steps of:
creating a virtual 3-D model of a patient's cranium and of the external soft tissue of the patient's head including at least one external anatomical structure by imaging equipment;
noting the location and trajectory of a fused cranial suture;
designing a virtual marking guide for placement on the soft tissue, the virtual marking guide comprising an upper portion with a window corresponding to the desired incision location to provide access to the fused cranial suture and a lower registration portion of the marking guide conforming to the at least one external anatomical structure; and
creating a physical marking guide based on the virtual marking guide, the physical marking guide comprising an elongated, strap-like, upper portion with a window corresponding to the desired incision location to provide access to the fused cranial suture and a lower registration portion of the marking guide conforming to the external anatomical structures,
whereby with the physical marking guide properly mounted to the external anatomical structure of the patient's head, the window is properly located over the fused cranial structure.

2. The method of claim 1, wherein the at least one external anatomical structure is an ear comprising a concha and a helix, and wherein the external soft tissue comprises temporal skin and a temporal junction between the helix and the temporal skin.

3. The method of claim 2, further comprising the step of configuring the lower registration portion of the physical marking guide to comprise an upper curved segment configured to be positioned on the patient along the temporal junction and a lower base segment configured to mate with the concha, such that the lower base segment has a concave configuration that matches the concave configuration of the concha.

4. The method of claim 2, wherein the at least one external anatomical structure are first and second ears, each ear comprising a concha and a helix, and wherein the external soft tissue comprises temporal skin and a temporal junction between the helix and the temporal skin.

5. The method of claim 4, further comprising the step of configuring a first and second lower registration portion of the physical marking guide, the first lower registration portion configured to comprise an upper curved segment configured to be positioned on the patient along the temporal junction of the first ear and a lower base segment configured to mate with the concha of the first ear, such that the lower base segment has a concave configuration that matches the concave configuration of the concha the first ear, and with the second lower registration portion configured to comprise an upper curved segment configured to be positioned on the patient along the temporal junction of the second ear and a lower base segment configured to mate with the concha of the second ear, such that the lower base segment has a concave configuration that matches the concave configuration of the concha the second ear.

6. A patient-specific craniosynostosis marking guide configured to be mounted onto an ear of a patient's head, the marking guide comprising:
an elongated, strap-like, upper portion having a window, the upper portion structured to conform to the patient's head;
a lower registration portion connected to the upper portion, the lower registration portion comprising a structured to abut the junction between the ear and temporal skin of the patient's head and comprising a convex lower base segment configured to match the size and configuration of the concave concha of the patient's ear when the marking guide is mounted on the patient's head.

7. The marking guide of claim 6, wherein the marking guide is configured to be mounted on a first and second ear of the patient's head, the marking guide further comprising a second lower registration portion connected to the upper portion, the second lower registration portion structured to abut the junction between the second ear and temporal skin of the patient's head and comprising a convex lower base segment configured to match the size and configuration of the concave concha of the patient's second ear when the marking guide is mounted on the patient's head.

8. A patient-specific ear-mountable craniosynostosis marking guide comprising:
an elongated, strap-like, upper portion having a window;
a lower registration portion connected to the upper portion, the lower registration portion comprising an upper curved segment and a convex lower base segment, the lower base segment configured to be receivable in mating manner within the concha of an ear.

9. The marking guide of claim 9, further comprising a second lower registration portion connected to the upper portion, the second lower registration portion comprising an upper curved segment and a convex lower base segment, the second lower base segment configured to be receivable in mating manner within the concha of an ear.
